(19) United States
(12) Patent Application Publication
(10) Pub. No.: US 2025/0145728 A1
(43) Pub. Date: May 8, 2025

(54) PREPARATION METHOD AND USE OF NON-NATURAL ANTI-HUMAN CD45RA MURINE CHIMERIC ANTIGEN RECEPTOR

(71) Applicants: Zhejiang University, Hangzhou City, Zhejiang Province (CN); Children's Hospital of Zhejiang University School of Medicine, Hangzhou City, Zhejiang Province (CN)

(72) Inventors: Yongmin TANG, Hangzhou City (CN); Ping ZHANG, Hangzhou City (CN); Yang TANG, Hangzhou City (CN)

(21) Appl. No.: 18/678,082

(22) Filed: May 30, 2024

(30) Foreign Application Priority Data
Nov. 2, 2023 (CN) 202311446355.9

(51) Int. Cl. C07K 16/28; A61K 38/00; A61P 35/02; C07K 14/705; C07K 14/725; C12N 5/0783; C12N 15/85; C12N 15/86

(52) U.S. Cl. CPC C07K 16/289; A61P 35/02; C07K 14/7051; C07K 14/70517; C12N 5/0636; C12N 15/85; C12N 15/86; A61K 38/00; C12N 2510/00; C12N 2740/15043

(57) ABSTRACT

The present disclosure provides a preparation method of a non-natural anti-human CD45RA murine chimeric antigen receptor (CAR). In the present disclosure, eukaryotic expression vectors pcDNA3.1/Mu3A4-4-1BB-3ζ and pcDNA3.1/Mu3A4-4-1BB-3ζ-EGFP and a lentiviral expression vector pLenti/Mu3A4-4-1BB-3ζ are constructed based on molecular biology using a mouse-derived 3A4 antibody. The lentiviral expression vector pLenti/Mu3A4-4-1BB-3ζ can successfully infect human T cells and effectively kill 3A4-positive target cells such as KG1a cells and Raji cells. The recognition of antigens by Mu3A4CAR does not depend on antigen presentation process and is not restricted by a major histocompatibility complex (MHC), thus overcoming tumor immune evasion to kill 3A4-positive tumor cells more effectively.

### CROSS REFERENCE TO RELATED APPLICATION

This patent application claims the benefit and priority of Chinese Patent Application No. 202311446355.9 filed with the China National Intellectual Property Administration on Nov. 2, 2023, the disclosure of which is incorporated by reference herein in its entirety as part of the present application.

### REFERENCE TO SEQUENCE LISTING

A computer readable XML file entitled “SEQUENCE LISTING”, that was created on Apr. 28, 2024, with a file size of about 19528 bytes, contains the sequence listing for this application, has been filed with this application, and is hereby incorporated by reference in its entirety.

### TECHNICAL FIELD

The present disclosure belongs to the field of biotechnology and mainly relates to a preparation method and use of a non-natural anti-human CD45RA murine chimeric antigen receptor (CAR). The present disclosure specifically relates to the design and preparation of a novel non-natural murine CAR gene (Mu3A4-CAR) for a murine anti-human leukocyte membrane antigen CD45RA novel target, and use thereof in production of a tumor drug preparation for targeted treatment of malignant tumors in the blood system using CD45RA-CAR gene-modified T cells (MuCD45RA-CAR-T cells).

### BACKGROUND

Leukemia is one of the most common hematological malignancies. Based on data collected in the United States from 2011 to 2015, leukemia incidence rates indicate 13.8 new cases and 6.7 leukemia-related deaths per 100,000 men and women annually. In recent decades, with the use of multiple chemotherapy drugs, the application of risk-stratified chemotherapy regimens, and the implementation of hematopoietic stem cell transplantation, the prognosis of leukemia has been significantly improved. However, the recurrence and drug resistance of leukemia remains the difficult problems in clinical treatment. It is reported that 20% of children with acute lymphoblastic leukemia (ALL) still face relapse, while the overall survival rate of adult ALL is only 30% to 40%. Cases of relapsed drug resistance lack effective alternative drugs, bringing great difficulties to clinical treatment.

In recent years, with the advancement of molecular biology and antibody engineering, targeted therapy for leukemia that only kills tumor cells without damaging normal cells has received widespread attention and development. Chimeric antigen receptor (CAR)-T cell therapy is one of the representatives. The targeted therapy of CAR-T is to design a CAR gene on autologous or allogeneic T cells to express the CAR fusion protein that recognizes a specific antigen target. The CAR gene is installed into human T cells through various biological transfection technologies, allowing them to continuously express a CAR fusion protein on the surface of T cells to produce a CAR-T cell preparation. The CAR-T cell preparation is then applied to selectively target and kill tumors with antigen targets by infusing CAR-T cells to exert clinical therapeutic effects. The CAR gene is an artificially-constructed fusion protein gene that contains an antigen recognition domain and a T cell signaling domain. T cells genetically engineered to express CAR can specifically recognize antigens and eliminate malignant tumors. The CAR-T therapy combines the specificity of targeted recognition for antigens in CAR antibodies and the potent killing effect mechanism of T cells to eliminate tumors in a major histocompatibility complex (MHC)-independent manner. CARs with different recognition domains have different targeting properties. For example, CD19 CAR mainly targets cells of the B cell lineage system, while CD33 CAR mainly targets cells of the myeloid cell lineage system. It can be seen that the recognition domain in the structure of a CAR gene (generally determined by the recognition characteristics of a single-chain fragment variable (scFv) region in the antibody structure) determines the targeted therapeutic use of the CAR gene. In recent years, CD19 CAR-T cell-targeted treatment of B-lineage tumors has been proven to achieve sustained disease remission and prolongation of survival time, and also brings new hope for the treatment of relapsed and refractory acute myeloid leukemia (AML) in children with CAR-T cells. However, compared with B-cell malignancies, the clinical trial results of this therapy in the treatment of AML are significantly worse than those in the treatment of ALL, making the most challenging task is how to select an ideal target molecule. Typically, target antigens present on the AML cell membrane are also highly expressed on the surface of normal myeloid cells. Therefore, the application of this kind of CAR-T cells in the treatment of relapsed and refractory AML may also cause severe neutropenia and easily lead to serious bacterial infections, thereby causing a devastating impact on the patient's health. Few AML targets have been approved for clinical treatment. So far, only CD123 CAR has been approved by the Food and Drug Administration (FDA) for the clinical treatment of relapsed and refractory AML, while other CAR targets including CD33, CD45, FLT3, Lewis-Y, and CLL-1 are still undergoing therapeutic trials and suffered from poor therapeutic effects. A crucial issue in CAR-T cell therapy research is to identify targets with strong specificity, so as to improve the targeted killing of tumor cells by CAR-T cells while minimizing the toxic side effects on normal tissue cells.

CD45 is a common antigen of leukocytes and a hematopoietic cell-specific tyrosine phosphatase. The CD45 is expressed on the membranes of all nucleated leukocytes in the hematopoietic system, but is not expressed on cells of the erythrocyte system, megakaryocyte system, hematopoietic stem cells, and other solid tissue cells. Alternative splicing of three exons 4(A), 5(B), and 6(C) in a CD45 antigen molecule can produce multiple isoforms. In order to confirm whether CD45 antibodies and their targets can be used to treat clinical patients, Peter Kletting conducted two measurement series with preloading and no preloading on 5 patients to obtain the biological distribution of ¹¹¹In-labeled anti-CD45 monoclonal antibodies under different saturation conditions. Biological distribution testing showed that the concentration in red bone marrow was significantly higher than that in important tissues such as liver, and optimal preloading increased bone marrow over-liver selectivity by 3.9 times (Kletting P, Kull T, Bunjes D, Luster M, Reske S N, Glatting G. Optimal preloading in radioimmunotherapy with anti-CD45 antibody. Medical physics. 2011; 38(5): 2572-2578.). Currently, radionuclide-labeled CD45 antibodies have been adopted internationally for conditioning management before hematopoietic stem cell transplantation. In a clinical trial, Pagel J M treated 58 patients with advanced AML or high-risk myelodysplastic syndrome using ¹³¹I-anti-CD45 antibody combined with fludarabine and 2 Gy total body irradiation in the conditioning regimen before allogeneic hematopoietic stem cell transplantation. The results showed that all patients achieved complete remission (Pagel J M, Gooley T A, Rajendran J, et al. Allogeneic hematopoietic cell transplantation after conditioning with ¹³¹I-anti-CD45 antibody plus fludarabine and low-dose total body irradiation for elderly patients with advanced acute myeloid leukemia or high-risk myelodysplastic syndrome. Blood. 2009, 114(27): 5444-5453.), indicating that CD45 antibodies could be used in humans without causing clinically unacceptable toxic side effects on other human tissue cells. However, the reactivity of ordinary CD45 antibodies with hematopoietic tissues is extremely broad, especially its expression occurs on the surface of all T cells, B cells, granulocytes, monocytes, NK cells, and DC cells. As a result, CD45 antibodies generally cannot be routinely used for targeted treatment of leukemia, thus avoiding severe cellular immune deficiency or neutrophil deficiency that may lead to severe bacterial and/or viral infections (Michelle L. Hermiston1, Zheng Xu, et al. CD45: A critical regulator of signaling thresholds in immune cells. Annu. Rev. Immunol. 2003, 21: 107-137.).

CD45RA, as an isoform of the CD45 molecule, is expressed on the surface of naive T cells, B cells, some granulocytes, and some monocytes, but is not expressed on activated T cells, memory T cells, mature red blood cells, platelets, and tissue cells of the body's parenchymal organs. Accordingly, this isoform does not cause anemia and thrombocytopenia as well as damages to other solid tissue cells (Li S, Tang Y, Zhang J, et al. 3A4, a new potential target for B and myeloid lineage leukemias [J]. J Drug Target, 2011, 19(9): 797-804.). Drug-resistant leukemia stem cells (LSCs) are thought to be responsible for relapse after the AML treatment. The CD45RA is expressed in leukemia cells in most AML patients, and the CD45RA serves as a specific marker for the AML-LSC cell subset (Kersten B, Valkering M, Wouters R, et al. CD45RA, a specific marker for leukaemia stem cell sub-populations in acute myeloid leukaemia. British journal of haematology. 2016, 173(2): 219-235.). Anti-CD45RA monoclonal antibodies can effectively target AML cells through their effector functions and apoptosis induction (Habibi-Anbouhi M, Kafi Z, Ghazizadeh L, et al. Cytotoxicity Assessment and Apoptosis-related Gene Profiling of Antibody Treated Acute Myeloid Leukemia (AML) and Acute Lymphocytic Leukemia (ALL) Cancerous Cell Lines. Iranian journal of allergy, asthma, and asthma immunology. 2019; 18(6): 679-687.). CD45RA does not respond to memory T cell subset (CD45RO+) cells that have been stimulated by antigens and have established immune activity against the antigens that the body has been exposed to, but only reacts with unstimulated T cell subsets (^{Naïve} T) (Tchilian E Z, Beverley P C. Altered CD45 expression and disease. Trends in immunology. 2006; 27(3): 146-153.). In view of this, when being used as a molecular targeted killing or treatment, the CD45RA should not destroy the established cellular immune function of the body. As Naïve T cells are cleared by CD45RA antibodies, the immune system of the body will lose part of its cellular immune response. However, after treatment is completed, normal hematopoietic stem cells can produce backup Naïve T cells to make up for this temporary low cellular immune function. As for the high expression of CD45RA on B cells, low humoral immune function can be compensated for by infusion of gamma globulin. In summary, CD45RA should be an ideal target for killing leukemia cells. The development of CAR-T cells targeting the CD45RA antigen can likely provide novel targeted therapeutic agents for the clinical treatment of leukemia.

### SUMMARY

A purpose of the present disclosure is to provide a non-natural anti-human CD45RA murine chimeric antigen receptor (CAR). The murine CAR is a CAR-T cell preparation that can recognize CD45RA antigen-positive leukemia cells, that is, a non-natural murine anti-human CD45RA-CAR gene-modified CAR-T cell. The CD45RA-CAR gene includes the following components: CD8a leader as a leading strand of the CAR, 3A4scFv as a target recognition domain (TRD), CD8a hinge as a hinge region, CD8a transmembrane as a CAR transmembrane domain (TMD), and an intracellular signal transduction region of 4-1BB (CD137) and an intracellular signal transduction region of CD3(connected in series to form an intracellular segment of the CAR. A murine 3A4scFv heavy-chain gene has a nucleotide sequence shown in SEQ ID NO: 3 and an amino acid sequence shown in SEQ ID NO: 4, while a murine 3A4scFv light-chain gene has a nucleotide sequence shown in SEQ ID NO: 5 and an amino acid sequence shown in SEQ ID NO: 6.

Based on an existing murine anti-human CD45RA immunoglobulin (ZCH-6-3A4 monoclonal antibody, Mu3A4 for short) gene sequence, a murine CAR (Mu3A4-CAR) gene against human CD45RA has been developed using molecular biology approaches, including the following steps:
(1) querying and comparing literatures to determine a basic structure of a second-generation 3A4CAR, retrieving the National Center for Biotechnology Information (NCBI) to verify gene sequences of a hinge region (hinge), a TMD, and an intracellular signal transduction region of the second-generation 3A4CAR, and synthesizing pUCI4-1BB-3ζ and pUC/4-1BB-3ζ-EGFP (enhanced green fluorescent protein) entrusted a company;
(2) constructing eukaryotic expression vectors pcDNA3.1/Mu3A4-4-1BB-3ζ and pcDNA3.1/Mu3A4-4-1BB-3ζ-EGFP and a lentiviral expression vector pLenti/Mu3A4-4-1BB-3ζ of the Mu3A4CAR gene through genetic engineering and molecular cloning; and
(3) conducting activity identification of the eukaryotic expression vectors and the lentiviral expression vector of the Mu3A4CAR gene: detecting whether the eukaryotic expression vectors and the lentiviral expression vector are expressed and whether protein localization is correct, comparing transfection and expression efficiencies of the eukaryotic expression vectors and the lentiviral expression vector, and selecting an optimal CAR expression vector.

The present disclosure provides two expression vectors, including CD8a leader (SEQ ID NO: 1)-CD45scFv (SEQ ID NO: 3+SEQ ID NO: 5)-CD8a hinge (SEQ ID NO: 7)-CD8a TMD (SEQ ID NO: 9)-CD137 (SEQ ID NO: 11)-CD3ζ (SEQ ID NO: 13) or CD8a leader (SEQ ID NO: 1)-3A4scFv (SEQ ID NO: 3+SEQ ID NO: 5)-CD8a hinge (SEQ ID NO: 7)-CD8a TMD (SEQ ID NO: 9)-CD137 (SEQ ID NO: 11)-CD3ζ (SEQ ID NO: 13), where the two expression vectors are an eukaryotic expression vector pcDNA3.1(+)-MuCD45RACAR or a pcDNA3.1(+)-Mu3A4CAR gene and a lentiviral expression vector pLenti-MuCD45RACAR or a pLenti-Mu3A4CAR gene, respectively.

Another purpose of the present disclosure is to provide use of the CAR gene (Mu3A4-CAR) in preparation of a drug for treating a disease mediated by CD45RA-expressing cells, that is, use in production of a pharmaceutical preparation for targeted treatment of a hematological malignant tumor with CD45RA-CAR gene-modified T cells (Mu3A4-CAR-T cells).

The disease refers to a tumor disease expressing a CD45RA membrane antigen, mainly a malignant hematological disease, specifically including acute myeloid leukemia, acute lymphoblastic leukemia, and malignant lymphoma; and the diseases further includes chronic myeloid leukemia and chronic lymphoblastic leukemia.

In the present disclosure, eukaryotic expression vectors pcDNA3.1/Mu3A4-4-1BB-3ζ and pcDNA3.1/Mu3A4-4-1BB-3ζ-EGFP and a lentiviral expression vector pLenti/Mu3A4-4-1BB-3ζ are constructed based on molecular biology using a murine 3A4 antibody. The lentiviral expression vector pLenti/Mu3A4-4-1BB-3ζ can successfully infect human T cells and effectively kill 3A4-positive target cells, KG1a cells and Raji cells. The recognition of antigens by Mu3A4CAR does not depend on antigen presentation, and is not restricted by a major histocompatibility complex (MHC), thus overcoming tumor immune evasion to kill 3A4-positive tumor cells more effectively.

In the present disclosure, a series of experiments are conducted using the Mu3A4CAR. After Mu3A4CAR successfully infected T cells, in vitro antigen-binding activity assay results show that the Mu3A4CAR-T can specifically bind to KG1a, a myeloid leukemia cell line that highly expresses CD45RA. The Mu3A4 CAR-T cells can target and kill leukemia cells in 3A4-positive cell lines and new AML patients. The Mu3A4 CAR-T cells can be used for the treatment of leukemias that highly express CD45RA antigen.

In the present invention, the recognition of antigens by the non-natural Mu3A4CAR protein does not depend on antigen presentation, and is not restricted by a major histocompatibility complex (MHC), thus overcoming tumor immune evasion to kill 3A4-positive tumor cells more effectively.

### BRIEF DESCRIPTION OF THE DRAWINGS

FIG. 1 shows a schematic structural diagram of the Mu3A4CAR gene; where CD8a leader is a leading strand of the CAR, Mu3A4scFv is a TRD, CD8a hinge is a hinge region, CD8a TM is a TMD of the CAR, 4-1BB is an intracellular costimulatory signal transduction region while CD3ζ is an intracellular signal transduction region, the above functional regions are connected in series to form a complete CAR gene of Mu3A4CAR; CD8a leader (NM_001768) has a sequence length of 51 bp, CD8a Hinge™ (NM_001768) has a sequence length of 207 bp, 4-1BB (NM_001561) has a sequence length of 126 bp, and CD3ζ (NM_198053) has a sequence length of 336 bp; Mu3A4scFv light-chain gene and heavy-chain gene come from the Patent ZL 2009 1 0246023.X;

FIGS. 2A-C shows schematic structural diagrams of the eukaryotic expression vectors pcDNA3.1/Mu3A4-4-1BB-3ζ (FIG. 2A) and pcDNA3.1/Mu3A4-4-1BB-3ζ-EGFP (FIG. 2B) and a lentiviral expression vector pLenti/Mu3A4-4-1BB-3ζ (FIG. 2C); where FIG. 2A is a non-fluorescent murine receptor (Murine), FIG. 2B is a murine receptor with EGFP fluorescence (EGFP-Murine), and a and b are transition vectors that can be used for transient expression; and FIG. 2C is a lentivirus murine receptor that is recombined into the cell genome and stably expressed at high levels, and can be used to infect T cells after lentivirus packaging, that is, CAR-T cells;

FIG. 3 shows the expression of Mu3A4CAR-EGFP fusion protein under an inverted fluorescence microscope (200× magnification); where EGFP green fluorescence can be observed under an inverted fluorescence microscope (b of FIG. 3) 24 h after pcDNA3.1/3A4CAR-EGFP infects CHO cells (a of FIG. 3); while no fluorescence is observed under the inverted fluorescence microscope (d of FIG. 3) 24 h after the empty vector is transfected into the CHO cells (c of FIG. 3), indicating that the 3A4CAR-EGFP fusion protein can be successfully expressed;

FIG. 4 shows the expression of fusion protein Mu3A4CAR-EGFP in CHO cells detected by cellular immunofluorescence method; a and i of FIG. 4 are bright field, b and j of FIG. 4 are blue field: DAPI; c and e of FIG. 4 are green fields: EGFP; d, f, g, and h of FIG. 4 are red fields: TRITC; and g, h, l of FIG. 4 are merge;

FIG. 5 shows that the pcDNA3.1/Mu3A4-4-1BB-3ζ eukaryotic expression vector detected by flow cytometry and the pLenti/Mu3A4-4-1BB-3ζ lentiviral expression vector can be successfully expressed in CHO cells, and the expression efficiency of the lentiviral expression vector pLenti/3A4CAR is higher than that of the eukaryotic expression vector pcDNA3.1/3A4CAR;

FIG. 6 shows that Mu3A4CAR, verified by flow cytometry, is successfully expressed on the surface of T cells, and a lentiviral supernatant of the Mu3A4CAR can infect T cells with a high efficiency (more than 40%) and can be expressed stably; where a of FIG. 6 is a scatter plot, b of FIG. 6 is a T cell group not infected with CAR, and c of FIG. 6 is a T cell group infected with Mu3A4CAR lentiviral vector; the cells in the infected group are distributed in 2 groups, one of which is Mu3A4CAR+ T cells and the other is ordinary T cells; a lentiviral supernatant of the Mu3A4CAR can infect T cells with a high efficiency (more than 40%) and can be expressed stably;

FIG. 7 shows the flow cytometric detection results of the binding of 3A4CAR-T to KG1a target cells with different effector-to-target ratios; where as an effector-to-target ratio increases, a ratio of the number of free KG1a to a number of bound KG1a (upper left quadrant/upper right quadrant) decreases sequentially; when the effector-to-target ratio reaches 5:1 or higher, not less than 70% of the target cells can be bound by 3A4CAR-T cells; and

FIG. 8 shows a killing effect of the Mu3A4CAR-T on 3A4(CD45RA)-positive leukemia cell lines; where KG1a is an acute myeloid leukemia cell line, Raji is a B-lineage lymphoma cell line, and Nalm-6 is a 3A4-negative B-lineage lymphoblastic leukemia cell line; and Mu3A4CAR-T cells have an obvious targeted killing effect on 3A4-positive leukemia/lymphoma cell lines in a dose-dependent manner, but have no obvious targeted killing effect on 3A4-negative Nalm-6 cells; and bars in the figure represent the mean t standard error.

### DETAILED DESCRIPTION OF THE EMBODIMENTS

The present disclosure will be further described with reference to the accompanying drawings and the examples.

### Example 1 the Nucleotide Sequence of a CAR Gene in the Present Disclosure

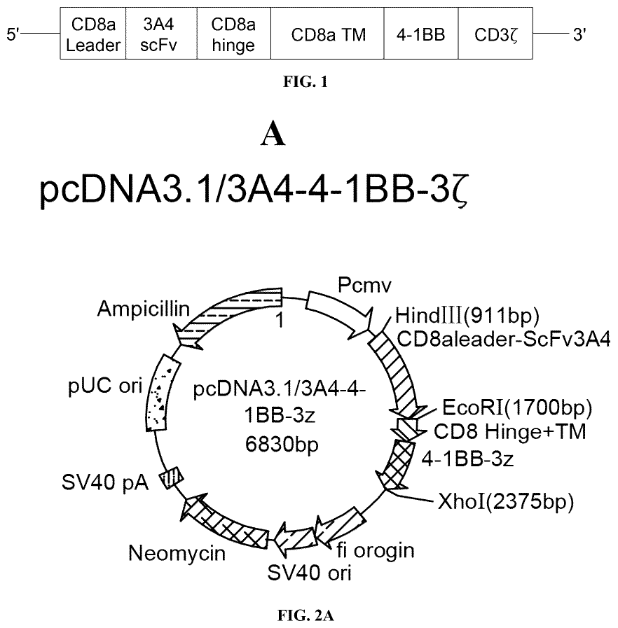

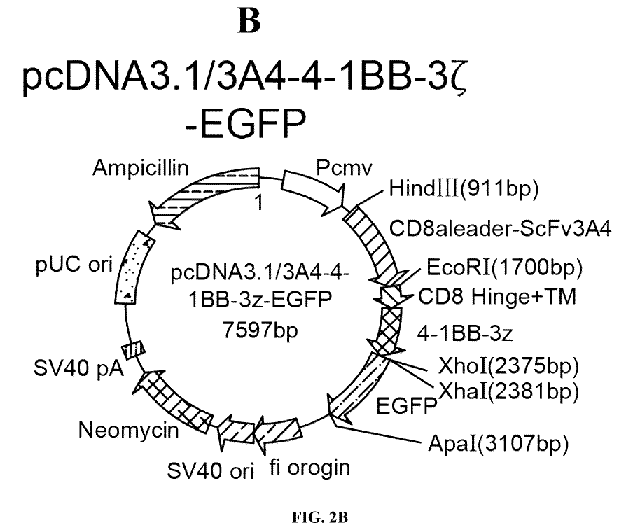

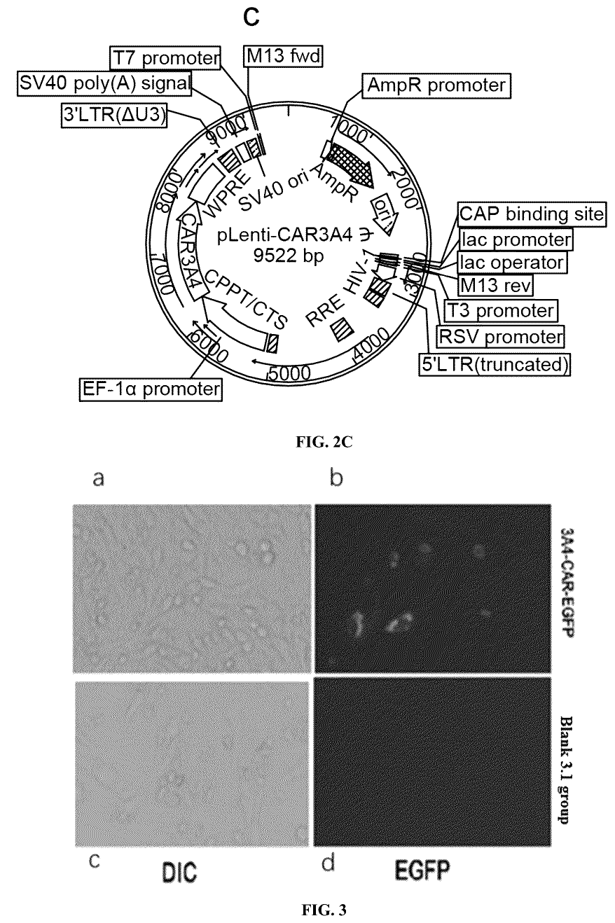

By querying literatures and the NCBI, a basic structure of Mu3A4CAR was established: CD8a leader was a leading chain of the CAR, Mu3A4scFv (single-chain antibody of murine 3A4) was a TRD, CD8a hinge was a hinge region, CD8a transmembrane was a TMD of the CAR, an intracellular signal transduction region of 4-1BB and an intracellular signal transduction region of CD3ζ were connected in series to form an intracellular segment of the CAR (FIG. 1). The partial sequences were verified through NCBI gene database comparison, among which CD8a leader (NM_001768) had a sequence length of 51 bp, CD8a Hinge™ (NM_001768) had a sequence length of 207 bp, 4-1BB (NM_001561) had a sequence length of 126 bp, and CD3ζ (NM_198053) had a sequence length of 336 bp. 3A4scFv light-chain gene and heavy-chain gene came from the Patent ZL 2009 10246023.X: a murine 3A4scFv heavy-chain gene had a nucleotide sequence shown in SEQ ID NO: 3 and an amino acid sequence shown in SEQ ID NO: 4, while a murine 3A4scFv light-chain gene had a nucleotide sequence shown in SEQ ID NO: 5 and an amino acid sequence shown in SEQ ID NO: 6.

### Example 2

The primers were designed based on the sequences of Mu3A4scFv and CD8a leader, PCR amplification was conducted on the Mu3A4scFv sequence containing CD8a leader, and Hind III and EcoR I restriction sites were added upstream and downstream, respectively. The upstream primer 3A4-leader P1 had a sequence(SEQ ID NO: 15): AAGCTTATGGCCTTACCAGTGACCGCCTTGCTCCTGCCGCTGGCCTTGCTGCTCCAC GCCGCCAGGCCGGCGGCCCAGCCGGCCCAG. The downstream primer 3A4-leader P2 had a sequence(SEQ ID NO: 16): GAATTCCCGTTTCAGCTCCAGCTTGG. PCR was conducted using pcDNA3.1/Hm3A4-His as a template: pre-denaturation at 95° C. for 5 min; denaturation at 94° C. for 30 sec, annealing at 60° C. for 30 sec and extension at 72° C. for 1 min and 30 sec, conducting 30 cycles in total; and extension repair at 72° C. for 10 min. The PCR reaction was terminated at 4° C. for 20 min. A target gene fragment CD8a-3A4 was purified by gel cutting, TA cloned, the target fragment was ligated to the pGEM®-T easy vector, the ligated product was transformed into competent bacteria DH5a, and then spread on an LB plate containing X-gal, IPTG, and 100 g/mL ampicillin, incubated in a constant-temperature water incubator at 37° C. overnight, a single well-separated, translucent, needle-tip-sized white colony was selected using a sterilized toothpick from the T-A clone blue and white screening plate, and placed in 7 mL of LB liquid medium containing 100 μg/mL ampicillin. The purified and extracted plasmid pGEM-T/CD8a-3A4 was labeled pGEM-T/CD8a-3A4.

Extraction and purification of plasmids pcDNA3.1/BB-3ζ (containing TAA), pcDNA3.1/BB-3ζ-EGFP, and pGEM-T/CD8a-3A4: after digestion with EcoR I and Hind III, respectively, the digested products were subjected to 1% agarose gel electrophoresis at 100 V for 30 min. The results were observed with a gel imaging system and the target gene fragment in the above agarose gel was recovered, and ligated with T4 ligase; the ligated product was transformed into competent cells DH5a, cloned and shaken bacteria were plated and amplified, plasmid was extracted, electrophoresed, and the bacterial solution was sequenced by a genetic company. A correctly sequenced product was added into bacterial solution mixed with 15% glycerol and frozen in a −80° C. refrigerator, and the plasmids were labeled pcDNA3.1/3A4-4-1BB-3ζ and pcDNA3.1/3A4-4-1BB-3ζ-EGFP, respectively.

Extraction and purification of plasmid pGEM-T/3A4-4-1BB-3ζ and pLenti: after digestion with Xbal I and Sal I, respectively, the digested products were subjected to 1% agarose gel electrophoresis at 100 V for 30 min. The results were observed with a gel imaging system and the target gene fragment in the above agarose gel was recovered. The ligation product was transformed into competent cells Trans1-blue, plated, cloned and shaken well to amplify, the plasmid was extracted and electrophoresed, and the bacteria solution was sequenced by a related genetic company. The correctly sequenced plasmid was labeled pLenti/3A4-4-1BB-3ζ and then frozen in a −80° C. refrigerator.

### Example 3

(1) Slide processing: ordinary coverslips were washed, soaked in acid overnight, washed with running water, baked, autoclaved, and then dried for later use.
(2) Chinese hamster ovary (CHO) cells in the logarithmic growth phase were digested with trypsin, and pipetted thoroughly to obtain a single cell suspension.
(3) A small amount of medium was added into a six-well plate, the slide was carefully placed in the plate, and the single cell suspension was added dropwise onto the slide such that 4×10⁵ cells were inoculated in each well. The plate was placed in an incubator at 37° C. with 5% CO₂ overnight.
(4) After 24 h, the cells grew on the coverslip and reached about 80% of confluence, and operations were conducted following instructions of Lipofectamine™ LTX and Plus: pcDNA3.1/3A4-4-1BB-3ζ-EGFP (pcDNA3.1/3A4-CAR-EGFP) and pcDNA3.1 plasmids were added to infect the CHO cells, and a negative control group (only adding Lipofectamine™ LTX and Plus), a pcDNA3.1 group (CHO/pcDNA3.1), and a pcDNA3.1/3A4-CAR-EGFP group (CHO/pcDNA3.1-3A4-CAR-EGFP) were set up.
(5) 24 h after transfection, the six-well plate was taken out of the 37° C. incubator to observe whether there was EGFP green fluorescence under an inverted fluorescent microscope; after 48 h to 72 h, the slides were removed from the six-well plate and rinsed three times with 1×PBS for 5 min each time.
(6) The cells were fixated in 4% paraformaldehyde at room temperature for 10 min and rinsed three times with 1×PBS for 5 min each time.
(7) The cells were permeabilized with 0.2% Triton X-100 for 5 min and rinsed three times with 1×PBS for 5 min each time. The cells were blocked with 4% normal goat serum for 30 min.
(8) The goat serum was discarded, the cells were washed three times with 1×PBS (5 min each time), added GAM Fab-TRITC (1:200 dilution) and kept in the wet box and incubated at 37° C. for 1 h, rinsed three times with PBS containing 1% Tween for 5 min each time.
(9) The cells were stained with DAPI for 1 min, and a drop of glycerol was added to cover the slide.
(10) The experimental results were observed under a fluorescence microscope and photographed.

EGFP green fluorescence could be observed under the inverted fluorescence microscope 24 h after infecting CHO cells with pcDNA3.1/3A4-CAR-EGFP (FIG. 3), initially indicating that the 3A4-CAR-EGFP fusion protein could be successfully expressed.

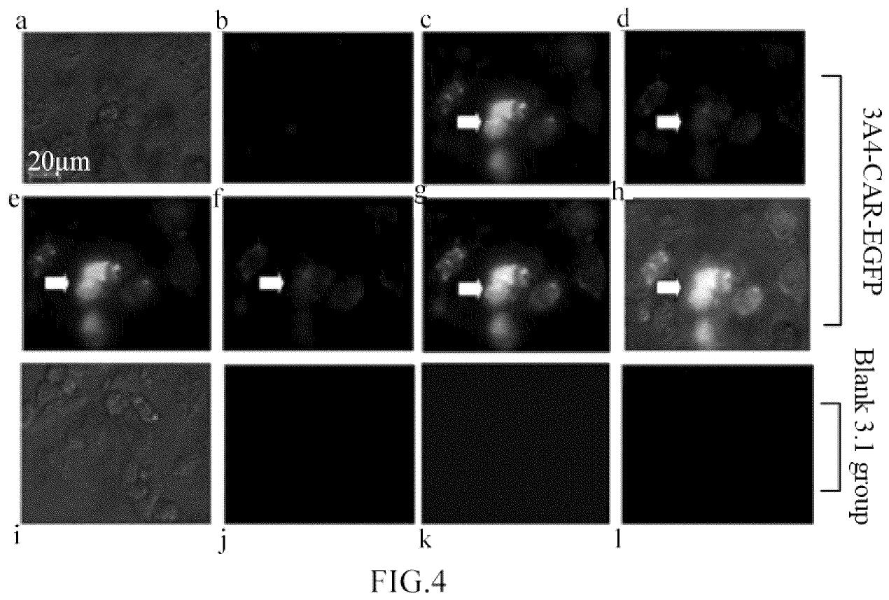

The results of cell immunofluorescence (FIG. 4) showed that in the transfected CHO cells: TRITC-stained 3A4-CAR expression could be seen in the cells and membranes (white arrows in d, f, g, h of FIG. 4); EGFP fluorescence expression could be seen in the cell (white arrows in c, e, g, h of FIG. 4); and DAPI-stained cell nuclei could be observed (b, c, d, h of FIG. 4). Moreover, the three colors could basically overlap completely. In CHO cells infected with the empty pcDNA3.1 plasmid, only DAPI-stained nuclei were visible.

### Example 4 Expression of pcDNA3.1/3A4-CAR and pLenti/3A4-CAR Proteins Detected by Flow Cytometry

(1) The CHO cells transfected for 3 d to 4 d were counted, the concentration was adjusted to 1×10⁶ cells/mL, 200 μL of cell suspension was taken from each flow tube, and the empty vector group, pcDNA3.1/3A4-CAR group, and pLenti/3A4CAR group were set up.
(2) Each group was equipped with 2 flow reaction tubes. The first tube was an isotype control, added with 4 μL of sheep IgG1-FITC; while 4 μL of GAM Fab IgG1-FITC was added to the second tube; the cells were incubated at 4° C. for 30 min.
(3) After incubation, the cells were washed with PBS, centrifuged at 1,000 rpm for 5 min each time, twice in total, and flow cytometric detection was conducted.

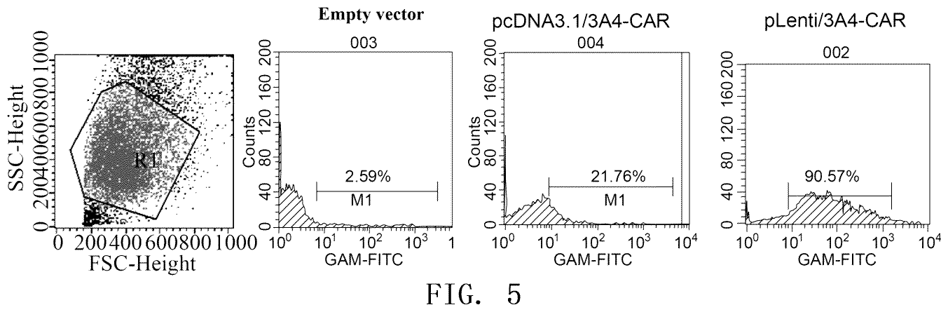

The results showed that (FIG. 5): both the lentiviral expression vector pLenti/3A4CAR and the eukaryotic expression vector pcDNA3.1/3A4CAR could successfully express the 3A4CAR recombinant protein in CHO cells; moreover, the expression efficiency of lentiviral expression vector pLenti/3A4CAR was higher than that of eukaryotic expression vector pcDNA3.1/3A4CAR (mean value: 90% v 23%; P<0.05).

### Example 5 Expression of 3A4CAR on T Cell Membrane Detected by Flow Cytometry

(1) 3A4CAR-T cells around the 7th day of infection and uninfected T cells of the same period were harvested. After cell counting, cells were collected by centrifugation at 1,000 rpm×5 min.
(2) 2 tubes were set up for each group of cells, with 2×10⁵ cells in each tube: the first tube was an isotype control with 4 μL of sheep IgG1 FITC added, while the second was an experimental tube with 4 μL of GAM-Fab-FITC added.
(3) The cells were incubated at 4° C. for 30 min in the dark.
(4) The cells were added with PBS and centrifuged at 1,000 rpm×5 min, washed twice, and detected by flow cytometry.

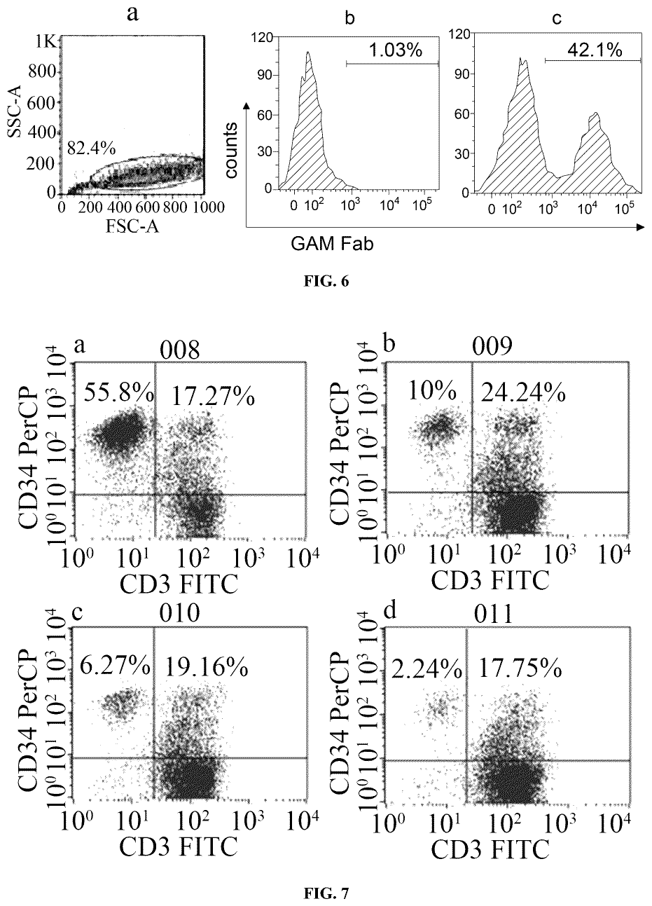

The results showed (FIG. 6): the cells in the infection group were distributed in two groups, one was 3A4CAR-positive T cells and the other was ordinary T cells. After many optimizations of virus packaging, T cell sorting, activation, infection, and culture processes, lentiviral supernatant packaged with 3A4CAR could infect T cells with a high efficiency (not less than 50%) and could be stably expressed.

### Example 6 Mu3A4CAR-T Specifically Killing Leukemia Cell Lines

(1) 3A4-positive KG1a and Raji, and 3A4-negative Nalm-6 were used as target cells, and divided into 3 groups for killing. Each group was set to have five concentration gradients with an effector-to-target ratio (E:T ratio) of 1:1, 2:1, 5:1, 10:1, and 16:1 separately. Under each E:T ratio gradient, the ordinary T cell group was set as the negative control well, and the Mu3A4CAR-T cell group was set as the experimental well.
(2) KG1a, Raji, and Nalm-6 cells in the logarithmic growth phase were counted, and a sufficient amount of target cells was taken according to the above experimental design.
(3) Calcein-AM was dissolved in DMSO (Dimethyl sulfoxide) to obtain a 1 mg/mL stock solution, and frozen in a −20° C. refrigerator. After diluting the Calcein-AM stock solution at 1:4000 with PBS, 10% a cell volume of the Calcein-AM diluted solution was added to the target cell suspension. After staining at 37° C. for 30 min, the cells were centrifuged and washed twice with RPMI1640 to remove excess dye.
(4) After centrifugation at 1,000 rpm for 5 min, a fresh RPMI1640 medium was added to adjust the cell concentrations of the three target cells to 10⁶ cells/mL, and 100 μL from each well was added to the corresponding well plate.
(5) Mu3A4CAR-T cells and T cells were prepared according to the effector-to-target ratio of 1:1, 2:1, 5:1, 10:1, and 16:1, centrifuged at 1,000 rpm×5 min, the magnetic beads were removed, the cells were resuspended in fresh RPMI1640 medium, and added to the target cells with the corresponding effector-to-target ratio. The cells were mixed by pipetting up and down.
(6) An appropriate amount of RPMI1640 complete medium was added to each well, and the cell concentration when incubating the mixture of effector cells and target cells was adjusted to about 1.5×10⁶ cells/mL.
(7) The cells were placed in a 48-well plate and incubated for 5 h in a dark incubator at 37° C. with 5% CO₂. The fluorescence intensity of the target cells was detected on a flow cytometer, and a same number of cells was collected from each tube.
(8) Killing rate=(fluorescence intensity of target cells in T cell group−fluorescence intensity of target cells in Mu3A4CAR-T cell group)/fluorescence intensity of target cells in T cell group.

As shown in FIG. 7, after 5 h of incubation, as the effector-to-target ratio gradually increased, the fluorescence intensity of Calcein-AM in the target cells gradually decreased. Specifically, the target cells KG1a were incubated with 3A4CAR-T at different effector-to-target (E:T) ratios. As the effector-to-target ratio increased, a ratio of the number of free KG1a to the number of bound KG1a (upper left quadrant/upper right quadrant) decreased successively, as follows: (a) E:T=1:1, ratio 3.24; (b) E:T=5:1, ratio 0.41; (c) E:T=10:1, ratio 0.243; (d) E:T=20:1, ratio 0.126.

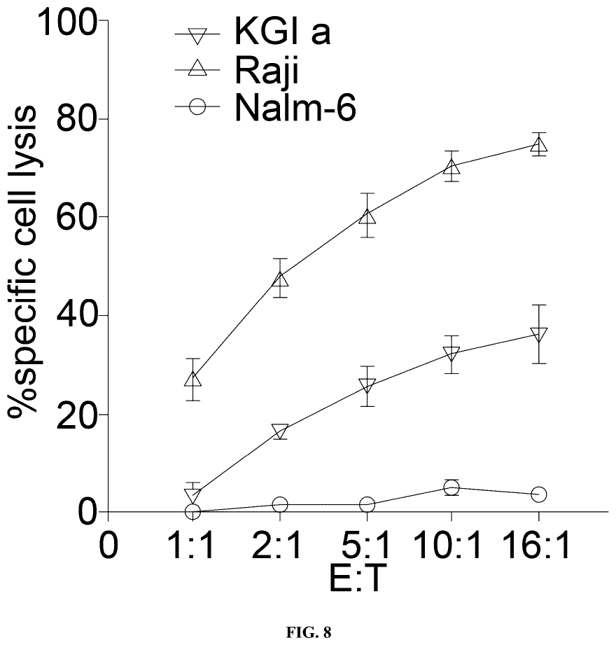

FIG. 8 showed the killing results: compared with uninfected T cells, Mu3A4CAR-T had a significant killing effect on both KG1a and Raji cells; as the effector-to-target ratio increased, the killing function could also increase; Mu3A4CAR-T had no obvious killing function against Nalm-6 cells that did not express 3A4 antigen.

What is claimed is:

1. A method for preparing a non-natural anti-human CD45RA murine chimeric antigen receptor (CAR), comprising: expressing a CAR protein through T cells and specifically binding the CAR protein to a human CD45RA antigen; wherein a target recognition domain (TRD) of the CAR is a human CD45RA antigen target; a MuCD45RACAR gene or a Mu3A4CAR gene comprises the following components: a CD8a leader serves as a leader sequence of the CAR, a murine CD45RA single-chain fragment variable (scFv) 3A4scFv serves as the TRD, a CD8a hinge serves as a hinge region, a CD8a transmembrane serves as a CAR transmembrane domain (TMD), and an intracellular signal transduction region of 4-1BB and an intracellular signal transduction region of CD3(are connected in series to form a complete murine CD45RACAR gene, namely the MuCD45RACAR gene or the Mu3A4CAR gene; a murine 3A4scFv heavy-chain gene has a nucleotide sequence shown in SEQ ID NO: 3 and an amino acid sequence shown in SEQ ID NO: 4, and a murine 3A4scFv light-chain gene has a nucleotide sequence shown in SEQ ID NO: 5 and an amino acid sequence shown in SEQ ID NO: 6.

2. The method according to claim 1, specifically comprising the following steps:
(1) querying and comparing literatures to determine a basic structure of a second-generation 3A4CAR, retrieving the National Center for Biotechnology Information (NCBI) to verify gene sequences of a hinge region, a TMD, and an intracellular signal transduction region of the second-generation 3A4CAR, and synthesizing pUC/4-1BB-3ζ and pUC/4-1BB-3ζ-EGFP;
(2) constructing eukaryotic expression vectors pcDNA3.1/Mu3A4-4-1BB-3ζ and pcDNA3.1/Mu3A4-4-1BB-3ζ-EGFP and a lentiviral expression vector pLenti/Mu3A4-4-1BB-3ζ of the Mu3A4CAR gene; and
(3) conducting activity identification of the eukaryotic expression vectors and the lentiviral expression vector of the Mu3A4CAR gene: detecting whether the eukaryotic expression vectors and the lentiviral expression vector are expressed and whether protein localization is correct, comparing transfection and expression efficiencies of the eukaryotic expression vectors and the lentiviral expression vector, and selecting an optimal CAR expression vector.

3. An effector cell, wherein the effector cell is a human T cell transfected through two expression vectors, namely a MuCD45RACAR-T cell or a Mu3A4CAR-T cell, wherein the two expression vectors comprises CD8a leader-CD45RAscFv-CD8a hinge-CD8a TMD-CD137-CD3ζ or CD8a leader-3A4scFv-CD8a hinge-CD8a TMD-CD137-CD3ζ, wherein the two expression vectors are an eukaryotic expression vector pcDNA3.1(+)-MuCD45RACAR or a pcDNA3.1(+)-Mu3A4CAR gene and a lentiviral expression vector pLenti-MuCD45RACAR or a pLenti-Mu3A4CAR gene, respectively.

4. A method for treating a disease mediated by cells expressing CD45RA, comprising administering the non-natural anti-human CD45RA murine CAR prepared by the method according to claim 1 to a subject in need thereof, wherein the disease refers to a tumor disease expressing a CD45RA membrane antigen, mainly a malignant hematological disease, specifically comprising acute myeloid leukemia, acute lymphoblastic leukemia, and malignant lymphoma; and the diseases further comprises chronic myeloid leukemia and chronic lymphoblastic leukemia.


SEQUENCE LISTING (16 sequences)

SEQ ID NO: 1 - DNA, 63 bp, synthetic construct
  source 1..63: mol_type = other DNA; note = CD8a leader
atggccttaccagtgaccgccttgctcctgccgctggccttgctgctccacgccgccaggccg

SEQ ID NO: 2 - protein, 21 aa, synthetic construct
  source 1..21: note = Signal peptide of CD8a leader
MALPVTALLLPLALLLHAARP

SEQ ID NO: 3 - DNA, 351 bp, synthetic construct
  source 1..351: mol_type = other DNA; note = 3A4scFv heavy-chain gene
caggtgcagctgaagcagtctggggctgaactggcaaaacctggggcctcagtgaagatgtcctgcaaggcttctggctccacctttactacctactggatacactgggtaaaacagaggcctggacagggtctggaatggattggatacattaatcctaacactggttatactgagtacaatcagaagttcaaggccaaggccacattgactgcagacaaatcctccagcacagcctacatgcaactgagcagcctgacatctgaggactctgcagtctattactgtgtaagatttattacggtagtaggggggtggggccaaggcaccactctcacagtctcctcagcc

SEQ ID NO: 4 - protein, 117 aa, synthetic construct
  source 1..117: note = 3A4scFv heavy-chain gene
QVQLKQSGAELAKPGASVKMSCKASGSTFTTYWIHWVKQRPGQGLEWIGYINPNTGYTEYNQKFKAKATLTADKSSSTAYMQLSSLTSEDSAVYYCVRFITVVGGWGQGTTLTVSSA

SEQ ID NO: 5 - DNA, 327 bp, synthetic construct
  source 1..327: mol_type = other DNA; note = 3A4scFv light-chain gene
caaattgttctcacccagtctccagcaatcatgtctgcatatctaggggaacgggtcaccatgacctgcactgccagttcaagtgtaagttccagtcacttgcactggtaccagcagaagccaggatcctcccccaaactctggatttatagcacatccaacctggcttctggagtcccagctcgcttcagtggcagtgggtctgggacctcttactctctcacaatcagcagcatggagactgaagatgctgccacttattactgccaccagtatcatcgttccccgctcacgttcggtgctgggaccaagctggagctgaaacgg

SEQ ID NO: 6 - protein, 109 aa, synthetic construct
  source 1..109: note = 3A4scFv light-chain gene
QIVLTQSPAIMSAYLGERVTMTCTASSSVSSSHLHWYQQKPGSSPKLWIYSTSNLASGVPARFSGSGSGTSYSLTISSMETEDAATYYCHQYHRSPLTFGAGTKLELKR

SEQ ID NO: 7 - DNA, 135 bp, synthetic construct
  source 1..135: mol_type = other DNA; note = CD8a hinge
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 8 - protein, 45 aa, synthetic construct
  source 1..45: note = CD8a hinge
TTTPAPRPPTPAPTIASQPLSLRPEACRPAAGGAVHTRGLDFACD

SEQ ID NO: 9 - DNA, 72 bp, synthetic construct
  source 1..72: mol_type = other DNA; note = CD8a transmembrane
atctacatctgggcgcccttggccgggacttgtggggtccttctcctgtcactggttatcaccctttactgc

SEQ ID NO: 10 - protein, 24 aa, synthetic construct
  source 1..24: note = CD8a transmembrane
IYIWAPLAGTCGVLLLSLVITLYC

SEQ ID NO: 11 - DNA, 126 bp, synthetic construct
  source 1..126: mol_type = other DNA; note = CD137 cytoplasmic domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 12 - protein, 42 aa, synthetic construct
  source 1..42: note = CD137 cytoplasmic domain
KRGRKKLLYIFKQPFMRPVQTTQEEDGCSCRFPEEEEGGCEL

SEQ ID NO: 13 - DNA, 336 bp, synthetic construct
  source 1..336: mol_type = other DNA; note = CD3z domain
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 14 - protein, 112 aa, synthetic construct
  source 1..112: note = CD3z domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 87 bp, synthetic construct
  source 1..87: mol_type = other DNA; note = upstream primer 3A4-leader P1
aagcttatggccttaccagtgaccgccttgctcctgccgctggccttgctgctccacgccgccaggccggcggcccagccggcccag

SEQ ID NO: 16 - DNA, 26 bp, synthetic construct
  source 1..26: mol_type = other DNA; note = downstream primer 3A4-leader P2
gaattcccgtttcagctccagcttgg